(19) United States
(12) Patent Application Publication
(10) Pub. No.: US 2006/0040895 A1
(43) Pub. Date: Feb. 23, 2006

(54) Aesthetic use of hyaluronan

(76) Inventor: Kipling Thacker, Excelsior, MN (US)

Correspondence Address: DORSEY & WHITNEY LLP;INTELLECTUAL PROPERTY DEPARTMENT, 50 SOUTH SIXTH STREET, MINNEAPOLIS, MN, 55402-1498, US

(21) Appl. No.: 11/205,400

(22) Filed: Aug. 17, 2005

Related U.S. Application Data
(60) Provisional application No. 60/602,904, filed on Aug. 19, 2004.

(51) Int. Cl. A61K 31/728

(52) U.S. Cl. 514/54

(57) ABSTRACT

A method to rejuvenate tissue by injection of a solution containing between about 0.5% and about 10% hyaluronic acid is described. The application of the solution to tissue regions having wrinkles, line, creases, abnormalities and the like is effective in diminishing the signs of aging.

### CROSS-REFERENCE TO RELATED APPLICATIONS

This application claims benefit under 35 U.S.C. § 119(e) to U.S. Ser. No. 60/602,904, filed on Aug. 19, 2004, entitled “Aesthetic Use Of Hyaluronan”, by Kipling Thacker (US Attorney Docket No. 34696/US), the contents of which are incorporated herein in their entirety for all purposes.

### BACKGROUND OF THE INVENTION

Human skin includes three skin layers; the top layer referred to as the epidermis, the second layer referred to as the dermis and the third layer referred to as subcutaneous fat. The dermis contains blood vessels, nerves, hair roots and sweat glands.

As we age, the levels of naturally occurring hyaluronan decrease in the skin. This leads to wrinkles, lines, dehydration and abnormalities in the skin. Higher amounts of hyaluronan slow the effects of the aging process down, however, there is no currently available method to increase production of hyaluronan in an individual.

There are many approaches to reduce lines, wrinkles and abnormalities of the skin that include topical applications or surgical treatment. There are many commercial products to help reduce the signs of aging. Generally, these include topical application of ointments, gels, oils or emulsions. Such products provide a hydrating effect to the skin, so that the skin softens, thus diminishing the line or wrinkle. However, the improvement to skin tone and/or softness is temporary. The effect(s) rapidly dissipate as the hydrating agent loses its potency or is removed during the normal course of the day.

A popular method to help reduce lines and wrinkles involves the injection of a neurotoxin. The use of neurotoxins for aesthetic purposes was first described in the medical literature around 1992. A well-known type of neurotoxin used in this application is botulinum toxin type A. Allergan Inc. sells botulinum toxin type A, useful in aesthetic applications under the brand name BOTOX®.

Typically, the neurotoxin is injected into the muscles in an amount that eases the wrinkles and creases and lasts for several months. A drawback with such treatment is that the injections leave the individual with a total inability to frown for the first 2.5 to 3 months. As the effect of the injections diminishes, the patient begins to develop more and more motion resulting in completely normal motion by six to seven months after injection.

Therefore, a need exists for skin application that helps alleviate signs of aging for a significant period of time without one or more of the aforementioned difficulties.

### BRIEF SUMMARY OF THE INVENTION

The present invention pertains to methods to rejuvenate an individual's skin tissue. This is accomplished by the sub-epidermal, i.e., intradermal, administration of a solution containing between about 0.5% and about 10% hyaluronic acid. Generally, the hyaluronic acid has a molecular weight range of between about 10,000 and about 8,000,000, or a range of between about 10,000 and 400,000, alternatively, between about 20,000 and about 100,000, and in an another embodiment, between about 400,000 and about 1,200,000. The solution is an aqueous solution that can include one or more salt, such as sodium salt(s), i.e., sodium chloride, potassium salt(s), i.e., potassium chloride, calcium salt(s), i.e., calcium chloride, magnesium salt(s), such as magnesium chloride, sodium acetate, sodium citrate and/or sodium phosphate. Alternatively, the solution can be a saline solution or PBS. In one aspect, the hyaluronic acid solution is delivered into the mid dermal region.

It should be understood, that the term “hyaluronic acid” includes hyaluronan. Hyaluronic acid, under physiological conditions, is converted into various forms, based on electrolytes and other physiological medium. Therefore, it should be understood that once the hyaluronic acid is placed in an electrolytic solution, it is more correctly known as hyaluronan.

Typically, the hyaluronic acid solution can be injected into the tissue area via a traditional needle syringe or by needleless syringe technology known in the art. The solution is administered in a sufficient amount to plump the skin surrounding the wrinkle or line (void) so that it is reduced to a point that it appears non-existent. Dependent upon the treatment site, one or more injections may be required, as determined by a skilled operator. The result is that the treated area reverts to a state where the skin appears youthful and healthy.

Advantageously, the present invention provides a method to decrease or eliminate fine lines, creases, wrinkles, abnormalities and the like, from the skin of an individual without causing paralysis to the muscle tissue/nerves in the surrounding area. More importantly, hyaluronic acid is a natural component of the skin, and therefore, does not present the problem of being rejected as a foreign body. This is in contrast to other materials, such as polyhydroxymethacrylic acid that is implanted into an area and could be rejected by the individual's physiological makeup.

Other features, utilities and advantages of various embodiments of the invention will be apparent from the following more particular description of embodiments of the invention as defined in the appended claims.

### DETAILED DESCRIPTION OF THE INVENTION

The present invention pertains to methods to rejuvenate an individual's skin tissue. This is accomplished, for example, by sub-epidermal, mid epidermal, or intradermal administration of a solution containing between about 0.5% and about 10% hyaluronic acid, a carboxyl-containing polysaccharide. More particularly, the solution contains between about 1% and about 5% hyaluronic acid, between about 2% and about 4%, and even more particularly, the solution contains between about 1% and about 3% hyaluronic acid.

The term “a carboxyl-containing polysaccharide” is intended to mean a polysaccharide containing at least one carboxyl group. The polysaccharide chosen may initially contain carboxyl groups or it may be derivatized to contain carboxyl groups. Examples of carboxyl-containing polysaccharides include, but are not limited to, carboxymethyl cellulose, carboxymethyl chitin, carboxymethyl chitosan, carboxymethyl starch, alginic acid, pectin, carboxymethyl dextran, and glucosaminoglycans such as heparin, heparin sulfate, chondroitin sulfate and hyaluronic acid (HA). In particular, useful carboxyl-containing polysaccharides include carboxymethyl cellulose, carboxymethyl chitin and HA. The most useful carboxyl-containing polysaccharide is HA.

The tissue rejuvenating solution includes the carboxyl-containing polysaccharide, or alternatively, a pharmacologically acceptable salt of the polysaccharide can be used, e.g., hyaluronan. Suitable pharmacologically acceptable salts are alkali or alkaline earth metal salts. In particular, the tissue rejuvenating solution employs the pharmacologically acceptable salt of the carboxyl-containing polysaccharide to prevent the formation of adhesions. Therefore, the tissue rejuvenating solution contains sodium hyaluronate.

Carboxyl-containing polysaccharides that can be used to prepare the tissue rejuvenating solution are known compounds that are described, for example, in U.S. Pat. No. 4,517,295 and U.S. Pat. No. 4,141,973; and Handbook of Water Soluble Gums and Resins, Chapter 4, by Stelzer & Klug, published by McGraw-Hill, 1980. Processes for preparing the most carboxyl-containing polysaccharide, HA, are illustrated in the Balazs patent, which details a procedure for extracting HA from rooster combs, and in U.S. Pat. No. 4,517,295 that describes fermentation process for making HA. The HA used to make the tissue rejuvenating solution should be highly purified (medical grade quality) for invasive applications.

Hyaluronic acid ionically crosslinked with polyvalent cations provides greater persistence in the injection site and helps prevent adhesions. Suitable agents that can be used to ionically crosslink the carboxyl-containing polysaccharide are compounds that possess polyvalent cations, such as divalent cations (Calcium salts) and in particular trivalent cations, e.g. ferric chloride, aluminum chloride, chromium sulfate, and aluminum sulfate. The most useful crosslinking agent is ferric chloride because of its low toxicity and biocompatibility with bodily tissue.

The concentration of polycationic species present in the reaction mixture should be a concentration sufficient to crosslink at least 10 percent of the carboxyl groups of the polysaccharide. In particular, the concentration of polycations will be sufficient to crosslink in the range of from about 60 to about 100 percent of the carboxyl groups of the polysaccharide and more particularly in the range of from 70 to 100 percent of the carboxyl groups of the polysaccharide and most particularly in the range of from 80 percent to 95 percent of the carboxyl group of the polysaccharide. Suitable methods to accomplish this are detailed in U.S. Pat. No. 5,532,221, the contents of which are incorporated herein in their entirety.

The weight average molecular weight of the HA is in the range of from about 10,000 to about 8,000,000. In some embodiments, the molecular weight of HA is between about 20,000 and about 100,000, in other embodiments the molecular weight of HA is between about 550,000 to about 8,000,000, more particularly in the range of from about 600,000 to about 2,000,000, still more particularly between about 400,000 and about 1,200,000, i.e., about 629,000 and about 1,060,000.

The tissue rejuvenating solution is an aqueous solution that includes sodium salt(s), i.e., sodium chloride, potassium salt(s), i.e., potassium chloride, calcium salt(s), i.e., calcium chloride, magnesium salt(s), such as magnesium chloride, sodium acetate, sodium citrate and/or sodium phosphate.

In one embodiment the hyaluronic acid containing solution, the tissue rejuvenating solution, is delivered via syringe to the tissue site. A unique aspect of the tissue rejuvenating solution of the invention is that the solution provides the operator with a consistent “delivery force” from syringe to syringe, injection to injection, and time and again. Not to be limited by theory, it is believed that the “delivery force” of the solution is related to the viscosity of the solution, geometry of the syringe, and internal diameter and length of the needle and its materials of construction.

The delivery of HA for facial aesthetics is important for proper correction of the tissue site. Delivery of a therapeutic, such as hyaluronic acid, via a needle depends upon the consistency of the product. A narrow gauge needle, about a 30 gauge needle, is most useful for precise delivery consistency and minimal pain upon injection. A product containing HA which is difficult to extrude or contains lumps will result in uneven delivery. At higher concentrations where HA is most effective, about 30 mg/ml, covalently crosslinked HA typically has an increased extrusion force. Since it is desirable to maintain a high concentration of HA, uncovalently crosslinked hyaluronic acid or hyaluronic acid that is ionically crosslinked provides good flow characteristics.

This “delivery force” or “expression force” is attributed to the amount of force required to push the tissue rejuvenating solution through a needle to the site. As stated above, it is believed that the “delivery” force is related to the viscosity of the solution. It is also believed that it is related to the length of the needle and the inner diameter of the needle.

The “delivery force” can be measured by Instron testing. For example, a 2.25 cc syringe filled with tissue rejuvenating solution and equipped with a 30-gauge needle is placed between the jaws of an Instron. The jaws are closed at a rate of 14 mm/minute with a result of between about 35 and about 56 Newtons, particularly between about 40 and about 45 Newtons of force to administer the solution. This is a suitable “delivery force” for an operator.

In another embodiment, a 1 cc syringe filled with tissue rejuvenating solution and equipped with a 30-gauge needle is also placed between the jaws of an Instron and tested as described above. The force required to expel the solution has an average extrusion value of between about 28 and about 33 Newtons.

Some current products have the disadvantage of causing “lumps” about the treated area. This is believed to be caused by inefficient delivery of the material to the treatment site. Not to be limited by theory it is considered that the heterogeneity of the current material(s) are responsible for the lack of uniformity during the treatment process. Recipients desire smooth skin after such a treatment and the occurrence of bumps, lumps, etc. does not meet that requirement. The present invention advantageously circumvents this phenomenon by the nature of the product. Again, not to be limited by theory, it is believed that the physical characteristics of the HA solution, the inherent viscosity of the solution, the selection of molecular weight of the HA and/or the concentration of the HA solution provide the advantage that lumps about the treatment area are avoided.

The present invention provides a hyaluronic acid containing solution that is shelf stable over a period of 2 to 3 years at a temperature between about 2 and about 30° C. without significant loss or increase in viscosity and thus the physical attributes that are attributable to the “delivery force”. This is an important advantage to the present tissue rejuvenating solution. The operator becomes accustomed to the “delivery force” of the required injection force and can administer the tissue rejuvenating solution to different individuals without the worry of not knowing if the solution will be delivered to quickly or too slowly to the injection site.

In another aspect, the viscosity of the tissue rejuvenating solution is between about 3.5×10⁴ to about 6.0×10⁴ cps at a concentration of between about 2.0 to about 4.0% of hyaluronic acid. In another aspect, the viscosity of the tissue rejuvenating solution is between about 4.2×10⁴ to about 5.2×10⁴ cps at a concentration of between about 2.7 to about 3.3% hyaluronic acid. In still another aspect, the kinetic viscosity of the tissue rejuvenating solution is between about 4.9×10⁴ to about 5.6×10⁴ cps at a shear rate of 2 seq⁻.

An “effective amount” of tissue rejuvenating solution applied to the site in need of rejuvenation, is an amount necessary to affect a reduction in the appearance of wrinkles, lines, creases or abnormalities in the tissue area. The amount applied will depend on numerous factors, most significantly of which is the surface area and/or void volume of the site. In particular, the amount of tissue rejuvenating solution applied should be enough to alter the tissue site such that the line, wrinkle, crease or abnormality is greatly diminished or is eliminated upon visual inspection. If necessary or desired, an additional amount sufficient to treat tissue adjacent to those areas can be injected to provide additional effect. The effective amount can be readily determined empirically.

The present invention provides an even more important advantage to the operator. The hyaluronic acid can be delivered via needleless injection. This method of delivery enjoys several advantages over needle bearing syringes. Hygienically, the delivery orifice of a needleless syringe can be sanitized quickly without the need to dispose of a needle. Otherwise, reuse of a needle provides an opportunity for infection amongst patients. There is generally less pain associated with needleless injection versus tradition injections that require a needle.

The term “needleless injection” is known in the art and is intended to encompass those methods and devices that provide an injection of a solution or solid under pressure, through a small aperture to a tissue site. The compressed gas, forces the liquid or solid through the epidermis, into the dermal region where treatment is desired. Suitable needleless injection apparatus include, for example, those described in U.S. Pat. Nos. 6,641,553, 6,099,504, 4,447,225, 6,746,429, 6,652,483, 6,641,554 and 5,520,639, the contents of which are incorporated in their entirety.

Although various embodiments of this invention have been described above with a certain degree of particularity, or with reference to one or more individual embodiments, those skilled in the art could make numerous alterations to the disclosed embodiments without departing from the spirit or scope of this invention. It is intended that all matter contained in the above description and shown in the accompanying drawings shall be interpreted as illustrative only of particular embodiments and not limiting. Changes in detail or structure may be made without departing from the basic elements of the invention as defined in the following claims.

What is claimed is:

1. A method to rejuvenate tissue, comprising the step of:
administering sub-epidermally to a subject in need thereof, a tissue rejuvenating solution containing between about 0.5% and about 10% hyaluronic acid, said hyaluronic acid having a molecular weight of between about 10,000 and about 8,000,000, wherein said tissue rejuvenating solution comprises an aqueous solution of a sodium salt, a potassium salt, a calcium salt, a magnesium salt, sodium acetate, sodium citrate, sodium phosphate or mixtures thereof.

2. The method of claim 1, wherein said hyaluronic acid has a molecular weight of between about 400,000 and about 1,200,000.

3. The method of claim 1, wherein said tissue rejuvenating solution is delivered into the intradermal region.

4. The method of claim 1, wherein said tissue rejuvenating solution is delivered via syringe containing a needle.

5. The method of claim 1, wherein said tissue rejuvenating solution is delivered via needleless injection.

6. The method of claim 1, wherein said hyaluronic acid molecular weight is between about 629,000 and about 1,060,000.

7. The method of claim 1, wherein the concentration of said hyaluronic acid is between about 1.0% and about 4.0%.

8. The method of claim 1, wherein the viscosity of the tissue rejuvenating solution is between about 3.5×10⁴ to about 6.0×10⁴ cps at a concentration of between about 2.0 to about 4.0% of hyaluronic acid.

9. The method of claim 1, wherein the viscosity of the tissue rejuvenating solution is between about 4.2×10⁴ to about 5.2×10⁴ cps at a concentration of between about 2.7 to about 3.3% hyaluronic acid.

10. The method of claim 1, wherein the shelf life stability of the tissue rejuvenating solution is between about 2 and about 3 years at a temperature range of between about 2 and about 30° C.

11. The method of claim 1, wherein said hyaluronic acid is sodium hyaluronate.

12. The method of claim 1, wherein said hyaluronic acid is not crosslinked.

13. The method of claim 12, wherein said crosslinking is not covalent.

14. The method of claim 12, wherein said crosslinking is not ionic.

15. The method of clam 1, wherein said hyaluronic acid is crosslinked.

16. The method of claim 15, wherein said hyaluronic acid is ionically crosslinked.

17. The method of claim 15, wherein said hyaluronic acid is covalently crosslinked.

18. The method of claim 1, wherein said application of said tissue rejuvenating solution is not topical.
